(19) United States
(12) Patent Application Publication
(10) Pub. No.: US 2012/0165635 A1
(43) Pub. Date: Jun. 28, 2012

(54) COMPENSATING FOR TEMPERATURE DRIFTS DURING GLUCOSE SENSING

(75) Inventors: Praveen Kumar Radhakrishnan, Singapore (SG); Shian Yeu Kam, Singapore (SG)

(73) Assignee: STMicroelectronics Asia Pacific Pte Ltd., Singapore (SG)

(21) Appl. No.: 12/975,810

(22) Filed: Dec. 22, 2010

(51) Int. Cl. A61B 5/1468

(52) U.S. Cl. 600/347

(57) ABSTRACT

Temperature variations in a patient's body can lead to inaccurate glucose readings. To compensate for changes in temperature, the temperature at a glucose sensing site can be sensed using a thermocouple. A compensated glucose level can be determined based on the temperature and the sensed glucose level. A glucose sensing device is described that includes a glucose sensor having a working electrode and a thermocouple having a junction positioned proximate the working electrode, with both the glucose and temperature sensors including the same metals.

### BACKGROUND

1. Technical Field

The techniques described herein relate to compensating for drifts in temperature at the site of glucose sensing.

2. Discussion of the Related Art

Various glucose sensing techniques are used for measuring the concentration of glucose in the blood. In one technique, known as amperometric glucose sensing, a reaction is initiated at a working electrode and a current measurement is made to sense the amount of glucose present. In some cases, a patient's blood glucose level can be measured on a continuous basis using a technique known as continuous glucose monitoring. Continuous glucose monitoring can be performed using the amperometric glucose sensing technique. To perform continuous glucose monitoring, a sensor can be implanted under the patient's skin, and a glucose measurement may be taken on a regular basis (e.g., every few minutes). The sensor may be implanted for several days to obtain information about the patient's glucose level over time.

### SUMMARY

Some embodiments relate to a glucose sensing device that includes a glucose sensor having a working electrode coated with an enzyme which selectively reacts with glucose molecules; and a thermocouple having a junction positioned proximate the working electrode.

Some embodiments relate to a glucose monitoring system that includes a glucose sensing device; a temperature sensor; and a glucose monitoring circuit that produces a compensated glucose measurement.

Some embodiments relate to a method of compensating for temperature variations in glucose sensing. A glucose level is measured at a sensing site. The temperature at the sensing site is measured. A compensated glucose level is determined based on the temperature and the sensed glucose level.

The foregoing summary of some embodiments is provided by way of illustration and is not intended to be limiting.

### BRIEF DESCRIPTION OF DRAWINGS

In the drawings, each identical or nearly identical component that is illustrated in various figures is represented by a like reference character. For purposes of clarity, not every component may be labeled in every drawing. The drawings are not necessarily drawn to scale, with emphasis instead being placed on illustrating various aspects of the invention.

FIG. 1A shows a plot of the current produced over time during amperometric glucose sensing, at different temperatures.

FIG. 1B shows a plot of the current produced during amperometric glucose sensing, at different temperatures.

FIG. 2A shows a diagram of a glucose sensing system, according to some embodiments.

FIG. 2B shows a cross section of the glucose sensing device of FIG. 2A.

FIG. 2C shows a diagram of a glucose sensing device having an alternative electrode configuration.

FIG. 3 shows a method of determining a temperature-compensated glucose measurement, according to some embodiments.

FIG. 4 shows a system for controlling a patient's blood glucose using an insulin pump, according to some embodiments.

### DETAILED DESCRIPTION

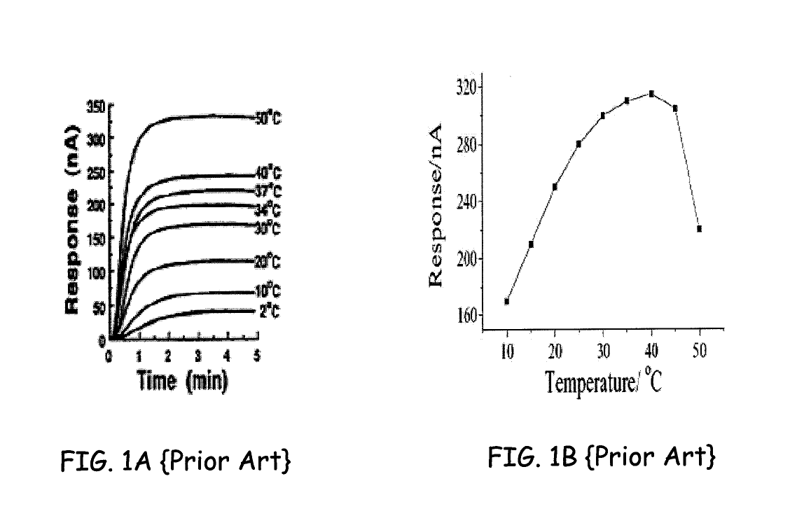

Continuous amperometric glucose sensing can be affected by temperature drifts within the patient's body. The temperature at the sensing site affects the speed of the reaction that takes place at the working electrode, which changes the amount of current produced. FIGS. 1A and 1B show plots of the current produced by amperometric glucose sensors at different temperatures. As shown in FIGS. 1A and 1B, the amount of current produced can vary significantly based on the temperature at the sensing site. Thus, the glucose reading varies based on the temperature at the sensing site.

The temperature within a patient's body can change due to factors such as patient physiology, patient activity, fever, or stress. The temperature at the sensing site can also be changed if the reaction at the sensing site is endothermic or exothermic, depending upon the sensing enzyme coated on the working electrode. Henceforth the combination of the working electrode and selectively reactive enzyme shall be referred to as the working electrode.

In some embodiments, the accuracy of the glucose reading can be improved by measuring the temperature within the patient's body at the site of the glucose sensor. A glucose sensing device is described that includes a thermocouple positioned proximate the glucose sensing site. Using a thermocouple can be particularly advantageous for continuous glucose monitoring applications because thermocouples do not require an external power source. The glucose measurement can then be compensated based on the temperature measurement to provide a more accurate glucose reading.

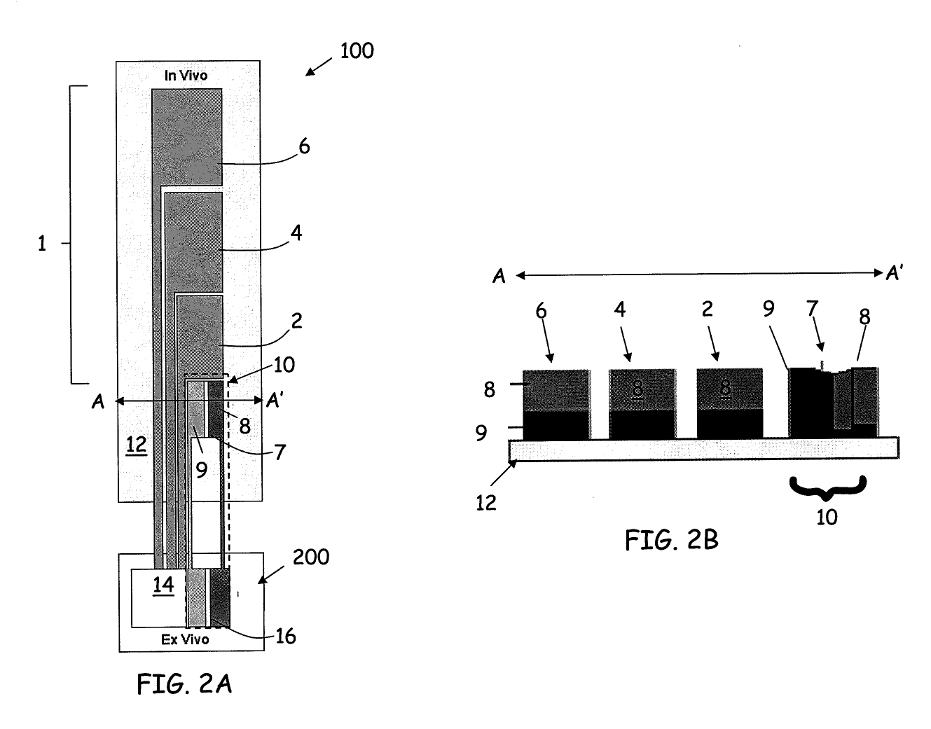

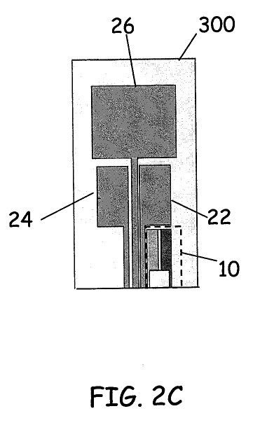

FIG. 2A shows a diagram of a glucose monitoring system that includes a glucose sensing device 100 connected to an external device 200 for signal processing, according to some embodiments. Glucose sensing device 100 includes a working electrode 2, a reference electrode 4 and a counter electrode 6. Electrodes 2, 4 and 6 form a portion of an amperometric glucose sensor 1 that may be used to perform continuous glucose monitoring. Glucose sensing device 100 also includes the hot junction 7 of a thermocouple 10 positioned proximate the working electrode 2 of the amperometric glucose sensor 1. In some embodiments, the hot junction 7 of the thermocouple 10 is positioned within approximately one millimeter or less of the working electrode 2 to provide better spatial accuracy for temperature measurement near the location of glucose sensing reaction. The thermocouple includes a first metal 8 and a second metal 9 that contact one another at the hot junction 7. The junction 7 of different metals 8 and 9, in combination with the cold junction 16, which is present in the external device 200, of the same thermocouple 10, causes a current to flow through the closed circuitry of the thermocouple 10. The voltage can be read across this circuit.

In some embodiments, the thermocouple 10 is a thin-film thermocouple formed by deposition of metals 8 and 9 with a region of overlap for the hot junction on the substrate 12 and another region of overlap for the cold junction in the external device 200. Also, depending on the performance on the thermocouple in the given application, the temperature sensing technology may be extended to a thermopile which comprises more than one thermocouple connected in series or parallel. Henceforth, the use of the term thermocouple also refers to the possible use of a thermopile. The use of a thin-film thermocouple can be advantageous because of its small thermal mass, which allows for a quicker response to changes in temperature than a bulk thermocouple. In some embodiments, the substrate 12 may be formed of a flexible, biocompatible material, such as polyimide. However, the techniques described herein are not limited in this respect, as any suitable material may be used for substrate 12. The working electrode 2, reference electrode 4, and counter electrode 6 of the amperometric glucose sensor 1 can be formed as metal thin films on the substrate 12. A suitable patterning process, such as photolithography, may be used to pattern the metal layer(s) to form electrodes 2, 4, and 6 and the thermocouple 10.

The glucose sensing device 100 is designed to be implanted within an organism, such as under the skin of a human body. When implanted, the glucose sensing device 100 can be used to perform continuous glucose monitoring. As shown in FIG. 1, glucose sensing device 100 is connected to an external device 200 that is configured to be positioned outside of the patient's body. The external device 200 is connected to the counter electrode 6, reference electrode 4 and working electrode 2 to obtain a glucose reading. The cold junction 16 is connected to the hot junction 7 to obtain a temperature reading. Glucose monitoring circuit 14 provides suitable signals to electrodes 2, 4 and 6 and receives signals therefrom to perform amperometric glucose sensing using such techniques as are known in the art, or compatible techniques which may be developed hereafter. In addition, glucose monitoring circuit 14 may be connected to receive one or more signals from the thermocouple 10, or the temperature compensation may be performed through the additional electronics in the external device 200. External device 200 includes a cold junction 16 between metals 8 and 9 that is used to produce a reference for thermocouple 10. Cold junction 16 may be cooled to and maintained at a temperature of 0° C., for example.

FIG. 2B shows a cross sectional view of the glucose sensing device 100 along the line A to A′ of FIG. 2A. As shown in FIG. 2B, the counter electrode 6, reference electrode 4 and working electrode 2 can be formed of two layers of metals. The first layer of metal can be an adhesion/seed layer 9, such as chromium, which is deposited on the substrate 12. The second layer of metal 8, such as inert, biocompatible gold, can be formed on the adhesion layer 9. In some embodiments, the metal layers forming the electrodes 2, 4, and 6 advantageously can be formed of the same metals 8, 9 that form the thermocouple 10. This can allow for greater simplicity and reduced costs in the manufacturing process, as electrodes 2, 4 and 6 and thermocouple 10 can be formed in the same manufacturing steps using the same materials, by eliminating the need for an extra target material for metal deposition and also eliminating the need for additional masks in the patterning process of the electrodes and the thermocouple. Metal 9 may be formed of a metal that is suitable for adhering to the material of substrate 12 and for forming a thermocouple with metal 8. For example, in some embodiments metal 9 may have a composition of approximately 90% nickel and approximately 10% chromium. However, this composition is provided by way of example, as metal 9 is not limited to a particular composition. Metal 8 may be formed of a metal that is resistant to corrosion so that it can form the top layers of electrodes 2, 4 and 6. In some embodiments metal 8 may have a composition of gold and approximately 0.07% iron. However, this composition is provided by way of example, as metal 8 is not limited to a particular composition.

After the step of forming electrodes 2, 4 and 6 and thermocouple 10, the device can be aged or annealed in a nitrogen atmosphere at a temperature of 400° C. for example, to prevent or limit a subsequent change in resistance of the metal layers. If a flexible substrate is used, such as polyimide, a lower annealing temperature may be used to avoid damaging the flexible substrate. In some embodiments, the thermocouple 10 can have a very wide temperature sensing range, accurate to within ±1° C. at 37° C.

FIG. 2C shows a diagram of a glucose sensing device 300 having an alternative electrode configuration. As shown in FIG. 2C, the working electrode 22, reference electrode 24, and counter electrode 26 are positioned in a different configuration from the configuration shown in FIG. 2A. Positioning the reference electrode 24 and working electrode 22 near each other may improve the accuracy of the glucose measurement. However, the techniques and devices described herein are not limited as to a particular electrode configuration, as any suitable electrode configuration may be used.

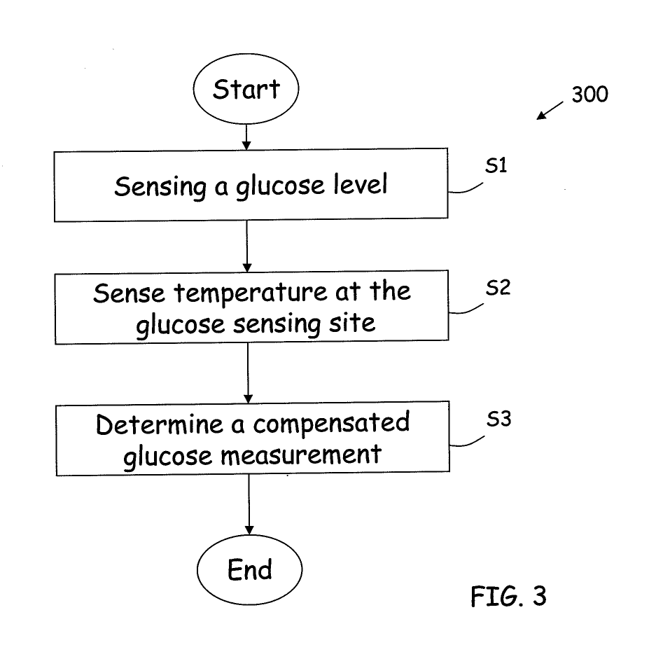

FIG. 3 shows a method of determining a temperature-compensated glucose measurement, according to some embodiments. In operation, the glucose monitoring circuit 14 is configured to determine a glucose measurement from the glucose sensing device 100 in step S1 and a temperature measurement from the thermocouple 10 in step S2. With the aid of the external device 200, steps S1 and S2 of 300 may be processed simultaneously. The glucose monitoring circuit then produces a compensated glucose measurement during step S3 based on the temperature measurement. The compensated glucose measurement may be produced in any suitable manner. In some embodiments, glucose monitoring circuit 14 can include a lookup table that includes glucose compensation values for different temperatures, which can be values tabulated from the patient's physiological history. The glucose monitoring circuit 14 can look up a glucose compensation value based on the temperature and then compensate the glucose measurement based on the glucose compensation value. For example, the glucose monitoring circuit can add or subtract the glucose compensation value to/from the glucose measurement to generate a compensated glucose measurement. In some embodiments, the glucose monitoring circuit may look up a glucose compensation value based on any suitable signal received from the thermocouple 10. A lookup table need not be used however, as any suitable technique may be used for mapping a measured temperature-dependent value to a compensated glucose measurement. The glucose compensation values may be determined based on the change in response over temperature as shown in FIGS. 1A-1B, or using any other suitable technique.

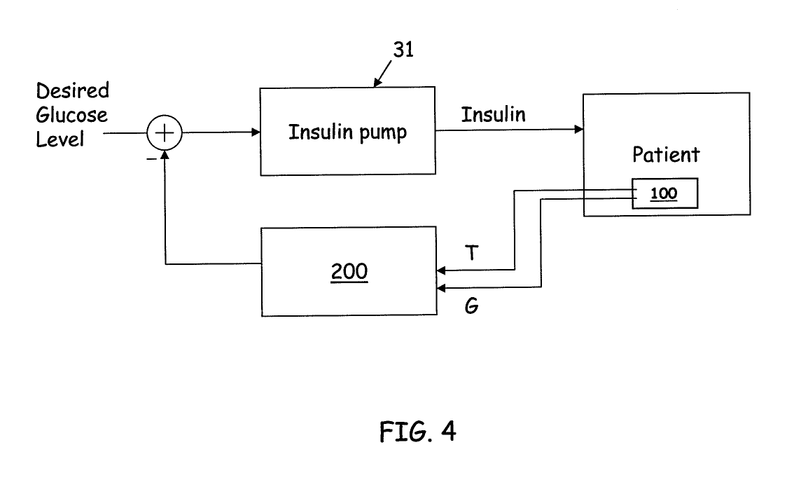

FIG. 4 shows a system level diagram of a system for controlling a patient's blood glucose using an insulin pump 31. In some embodiments, the techniques described herein may be used for providing a signal to control an insulin pump that regulates a patient's blood glucose level. The amount of insulin provided to the patient by the insulin pump is controlled based on the glucose measurement G and the temperature measurement T. As discussed above, the temperature-compensated glucose measurement produced using external device 200 and can be used to control the insulin pump 31. A control circuit can compare the temperature-compensated glucose level with a desired glucose level so that a quantity of insulin is provided to the patent based on the difference between the desired and measured glucose levels. Thus, the patient's blood glucose level can be controlled using feedback. However, any suitable control techniques may be used to control the insulin pump, as the techniques described herein are not limited to a particular control technique.

The above-described embodiments and others can be implemented in any of numerous ways. For example, any of the components of glucose monitoring circuit 14 and/or external device 200 may be implemented using hardware, software or a combination thereof. When implemented in hardware, any suitable hardware may be used, such as general-purpose or application-specific hardware. For example, external device 200 can be implemented using an application specific integrated circuit (ASIC). When implemented in software, the software code can be executed on any suitable hardware processor or collection of hardware processors, whether provided in a single computer or distributed among multiple computers.

Some embodiments include at least one tangible non-transitory computer-readable storage medium (e.g., a computer memory, a floppy disk, an optical disk, a tape, etc.) encoded with a computer program (i.e., a plurality of instructions), which, when executed on a processor, perform the above-discussed functions. In addition, it should be appreciated that the reference to a computer program which, when executed, performs the above-discussed functions, is not limited to an application program running on a host computer. Rather, the term computer program is used herein in a generic sense to reference any type of computer code (e.g., software or microcode) that can be employed to program a processor to implement the above-discussed aspects of the techniques described herein.

This invention is not limited in its application to the details of construction and the arrangement of components set forth in the foregoing description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

What is claimed is:

1. A glucose sensing device, comprising:
a glucose sensor comprising a working electrode; and
a thermocouple having a hot junction positioned proximate the working electrode.

2. The glucose sensing device of claim 1, wherein the glucose sensor comprises an amperometric glucose sensor and the amperometric glucose sensor is configured to perform a glucose sensing reaction at the working electrode.

3. The glucose sensing device of claim 1, wherein the hot junction is positioned within approximately one millimeter of the working electrode.

4. The glucose sensing device of claim 1, wherein the thermocouple further comprises a cold junction.

5. The glucose sensing device of claim 1, wherein the thermocouple comprises a thin-film thermocouple.

6. The glucose sensing device of claim 5, further comprising a substrate on which are formed the thin-film thermocouple and the glucose sensor.

7. The glucose sensing device of claim 6, wherein the substrate is a flexible substrate.

8. The glucose sensing device of claim 7, wherein the flexible substrate comprises polyimide.

9. The glucose sensing device of claim 1, wherein the thermocouple comprises a first metal and a second metal, both being dissimilar.

10. The glucose sensing device of claim 9, wherein the working electrode comprises the first metal.

11. The glucose sensing device of claim 10, wherein the working electrode further comprises the second metal.

12. The glucose sensing device of claim 9, wherein the first metal comprises nickel and chromium.

13. The glucose sensing device of claim 12, wherein the composition of the first metal is approximately 10% chromium and approximately 90% nickel.

14. The glucose sensing device of claim 9, wherein the second metal comprises gold.

15. The glucose sensing device of claim 14, wherein the second metal further comprises iron.

16. The glucose sensing device of claim 15, wherein the first metal comprises nickel and chromium.

17. The glucose sensing device of claim 15, wherein a composition of the second metal comprises gold and 0.07% iron.

18. The glucose sensing device of claim 17, wherein a composition of the first metal is approximately 10% chromium and 90% nickel.

19. A glucose monitoring system, comprising:
a glucose sensing device;
a temperature sensor; and
a glucose monitoring circuit configured to produce a compensated glucose measurement.

20. The glucose monitoring system of claim 19, wherein the temperature sensor comprises a thermocouple.

21. The glucose monitoring device of claim 19, wherein the thermocouple and glucose sensing electrodes of the glucose sensing device comprise the same metal, deposited on the same seed layer.

22. The glucose monitoring device of claim 19, wherein the cost of manufacturing is reduced by reducing the number of metals used.

23. A method, comprising:
sensing a glucose level at a sensing site;
sensing a temperature at the sensing site; and
determining a compensated glucose level based on the temperature and the sensed glucose level.

24. The method of claim 23, wherein the temperature is measured using a thermocouple.

25. The method of claim 23, further comprising:
controlling an insulin pump using the compensated glucose level.
